(19) United States
(12) Patent Application Publication
(10) Pub. No.: US 2025/0309346 A1
(43) Pub. Date: Oct. 2, 2025

(54) ELECTROLYTE FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: SUNDAE KIM, Yongin-si (KR); Yeji YANG, Yongin-si (KR); SANGHOON KIM, Yongin-si (KR); Dahyun KIM, Yongin-si (KR); Sangwoo PARK, Yongin-si (KR); Hongryeol PARK, Yongin-si (KR); HYUNBONG CHOI, Yongin-si (KR)

(21) Appl. No.: 18/916,551

(22) Filed: Oct. 15, 2024

(30) Foreign Application Priority Data
Mar. 28, 2024 (KR) 10-2024-0042707

(51) Int. Cl. H01M 10/0567; C07D 271/06; H01M 4/02; H01M 4/36; H01M 4/505; H01M 4/525; H01M 4/583; H01M 10/0568; H01M 10/0569

(52) U.S. Cl. CPC H01M 10/0567; C07D 271/06; H01M 4/362; H01M 4/505; H01M 4/525; H01M 4/583; H01M 10/0568; H01M 10/0569; H01M 2004/027; H01M 2004/028; H01M 2220/30; H01M 2300/004

(57) ABSTRACT

Disclosed are electrolytes and rechargeable lithium batteries including the same. The electrolyte for a rechargeable lithium battery comprises a non-aqueous organic solvent, a lithium salt, and an additive represented by Chemical Formula 1 discussed in this disclosure.

### CROSS-REFERENCE TO RELATED APPLICATION

The present application claims priority to and the benefit of Korean Patent Application No. 10-2024-0042707 filed on Mar. 28, 2024, in the Korean Intellectual Property Office, the entire content of which is hereby incorporated by reference.

### BACKGROUND

### 1. Field

Embodiments of the present disclosure relate to an electrolyte for a rechargeable lithium battery and a rechargeable lithium battery including the same.

### 2. Description of Related Art

Recently, with the rapid spread of battery using electronic devices, such as mobile phones, laptop computers, and electric vehicles, there is a rapidly increasing demand for rechargeable batteries having high energy density and high capacity. Therefore, intensive research has been conducted to improve performance of rechargeable lithium batteries.

A rechargeable lithium battery includes a positive electrode, a negative electrode, and an electrolyte, which positive and negative electrodes include an active material in which intercalation and deintercalation of lithium are possible, and generates electrical energy caused by oxidation and reduction reactions if lithium ions are intercalated and deintercalated.

A lithium salt dissolved in a non-aqueous organic solvent is used as the electrolyte of the rechargeable lithium battery. Characteristics of the rechargeable lithium battery are exhibited by complex reactions between the positive electrode and the electrolyte and between the negative electrode and the electrolyte. Accordingly, the use of a suitable or appropriate electrolyte is one variable for improvement of the rechargeable lithium battery.

### SUMMARY

An embodiment of the present disclosure provides an electrolyte for a rechargeable lithium battery having improved stability and lifetime characteristics at high temperatures.

An embodiment of the present disclosure provides a rechargeable lithium battery including the electrolyte.

According to an embodiment of the present disclosure, an electrolyte for a rechargeable lithium battery may include: a non-aqueous organic solvent; a lithium salt; and an additive represented by Chemical Formula 1.

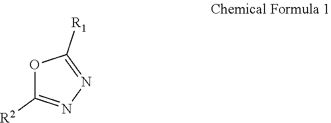

In Chemical Formula 1, R₁ may be a C1 to C10 alkyl group substituted with one or more fluorine atoms, and R₂ may be a C6 to C15 aryl group.

According to an embodiment of the present disclosure, a rechargeable lithium battery may include: a positive electrode that includes a positive electrode active material; a negative electrode that includes a negative electrode active material; and the electrolyte for the rechargeable lithium battery.

### BRIEF DESCRIPTION OF DRAWINGS

The accompanying drawings, together with the specification, illustrate embodiments of the subject matter of the present disclosure, and, together with the description, serve to explain principles of embodiments of the subject matter of the present disclosure.

FIG. 1 is a simplified conceptual diagram showing a rechargeable lithium battery according to an embodiment of the present disclosure.

FIGS. 2-5 are simplified diagrams showing rechargeable lithium batteries according to embodiments of the present disclosure.

FIGS. 6-7 are graphs showing results of cyclic voltammetry (CV) according to Embodiment 1 and Comparative Example 1.

### DETAILED DESCRIPTION

In order to sufficiently understand the configuration and effect of the subject matter of the present disclosure, some embodiments of the present disclosure will be described with reference to the accompanying drawings. It should be noted, however, that the present disclosure is not limited to the following example embodiments, and may be implemented in various suitable forms. Rather, the example embodiments are provided only to disclose the subject matter of the present disclosure and let those having ordinary skill in the art fully know the scope of the present disclosure.

In this description, it will be understood that, if an element is referred to as being on another element, the element can be directly on the other element or intervening elements may be present between therebetween. In the drawings, thicknesses of some components may be exaggerated for effectively explaining the technical contents of the present disclosure. Like reference numerals refer to like elements throughout the specification.

Unless otherwise specially noted in this description, the expression of a singular form may include the expression of a plural form. In embodiments, unless otherwise specially noted, the phrase “A or B” may indicate “A but not B”, “B but not A”, and “A and B”. The terms “comprises/includes” and/or “comprising/including” used in this description do not exclude the presence or addition of one or more other components.

As used herein, the term “combination thereof” may refer to a mixture, a stack, a composite, a copolymer, an alloy, a blend, and/or a reaction product.

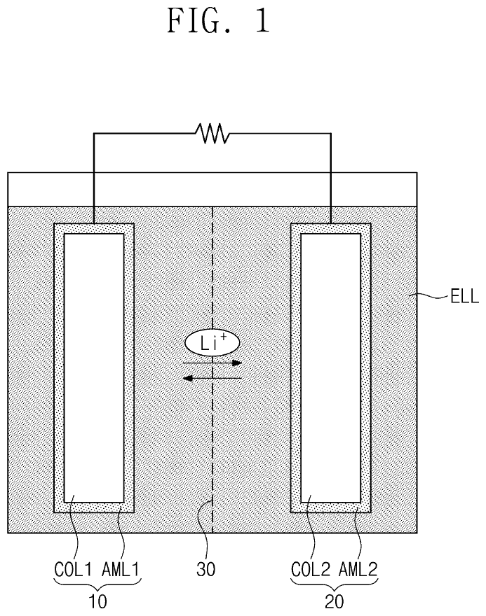

FIG. 1 is a simplified conceptual diagram showing a rechargeable lithium battery according to an embodiment of the present disclosure. Referring to FIG. 1, a rechargeable lithium battery may include a positive electrode 10, a negative electrode 20, a separator 30, and an electrolyte ELL.

The positive electrode 10 and the negative electrode 20 may be spaced apart from each other across the separator 30. The separator 30 may be between the positive electrode 10 and the negative electrode 20. The positive electrode 10, the negative electrode 20, and the separator 30 may be in contact with the electrolyte ELL. The positive electrode 10, the negative electrode 20, and the separator 30 may be impregnated with the electrolyte ELL.

The electrolyte ELL may be a medium by which lithium ions are transferred between the positive electrode 10 and the negative electrode 20. In the electrolyte ELL, the lithium ions may move through the separator 30 toward one of the positive electrode 10 or the negative electrode 20.

### Positive Electrode 10

The positive electrode 10 for a rechargeable lithium battery may include a current collector COL1 and a positive electrode active material layer AML1 formed on the current collector COL1. The positive electrode active material layer AML1 may include a positive electrode active material and further include a binder and/or a conductive material (e.g., an electrically conductive material).

For example, the positive electrode 10 may further include an additive that can serve as a sacrificial positive electrode.

An amount of the positive electrode active material may be about 90 wt % to about 99.5 wt % relative to 100 wt % of the positive electrode active material layer AML1. An amount of each of the binder and the conductive material may be about 0.5 wt % to about 5 wt % relative to 100 wt % of the positive electrode active material layer AML1.

The binder may serve to improve attachment of positive electrode active material particles to each other and also to improve attachment of the positive electrode active material to the current collector COL1. The binder may include, for example, polyvinyl alcohol, carboxymethyl cellulose, hydroxypropyl cellulose, diacetyl cellulose, polyvinylchloride, carboxylated polyvinylchloride, polyvinylfluoride, ethylene oxide-containing polymer, polyvinylpyrrolidone, polyurethane, polytetrafluoroethylene, polyvinylidene fluoride, polyethylene, polypropylene, styrene-butadiene rubber, (meth)acrylated styrene-butadiene rubber, epoxy resin, (meth)acrylic resin, polyester resin, and/or nylon, but the present disclosure is not limited thereto.

The conductive material may be used to provide an electrode with conductivity (e.g., electrical conductivity), and any suitable conductive material that does not cause a chemical change of a battery (e.g., that does not cause an undesirable chemical change in the rechargeable lithium battery) may be used as the conductive material to constitute the battery. The conductive material may include, for example, a carbon-based material such as natural graphite, artificial graphite, carbon black, acetylene black, Ketjenblack, carbon fiber, carbon nano-fiber, and/or carbon nano-tube; a metal powder and/or metal fiber containing one or more of copper, nickel, aluminum, and/or silver; a conductive polymer (e.g., an electrically conductive polymer) such as a polyphenylene derivative; or a mixture thereof.

Aluminum (Al) may be used as the current collector COL1, but the present disclosure is not limited thereto.

### Positive Electrode Active Material

The positive electrode active material in the positive electrode active material layer AML1 may include a compound (e.g., lithiated intercalation compound) that can reversibly intercalate and deintercalate lithium. For example, the positive electrode active material may include at least one kind of composite oxide including lithium and metal that is selected from cobalt, manganese, nickel, and a combination thereof.

The composite oxide may include lithium transition metal composite oxide, for example, lithium-nickel-based oxide, lithium-cobalt-based oxide, lithium-manganese-based oxide, lithium-iron-phosphate-based compounds, cobalt-free nickel-manganese-based oxide, or a combination thereof.

For example, the positive electrode active material may include a compound represented by one selected from the chemical formulae below. LiₐA_{1-b}X_{b}O_{2-c}D_{c} (where 0.90≤a≤1.8, 0≤b≤0.5, and 0≤c≤0.05); LiₐMn_{2-b}X_{b}O_{4-c}D_{c} (where 0.90≤a≤1.8, 0≤b≤0.5, and 0≤c≤0.05); LiₐNi_{1-b-c}Co_{b}X_{c}O_{2-α}D_{α} (where 0.90≤a≤1.8, 0≤b≤0.5, 0≤c≤0.5, and 0<α<2); LiₐNi_{1-b-c}Mn_{b}X_{c}O_{2-α}D_{α} (where 0.90≤a≤1.8, 0≤b≤0.5, 0≤c≤0.5, and 0<α<2); LiₐNi_{b}Co_{c}L¹_{d}GeO₂ (where 0.90≤a≤1.8, 0≤b≤0.9, 0≤c≤0.5, 0≤d≤0.5, and 0≤e≤0.1); LiₐNiG_{b}O₂ (where 0.90≤a≤1.8 and 0.001≤b≤0.1); LiₐCoG_{b}O₂ (where 0.90≤a≤1.8 and 0.001≤b≤0.1); LiₐMn_{1-b}G_{b}O₂ (where 0.90≤a≤1.8 and 0.001≤b≤0.1); LiₐMn₂GbO₄ (where 0.90≤a≤1.8 and 0.001≤b≤0.1); LiₐMn_{1-g}G_{g}PO₄ (where 0.90≤a≤1.8 and 0≤g≤0.5); Li_{(3-f)}Fe₂(PO₄)₃ (where 0≤f≤2); and LiₐFePO₄ (where 0.90≤a≤1.8).

In the chemical formulae above, A is Ni, Co, Mn, or a combination thereof, X is Al, Ni, Co, Mn, Cr, Fe, Mg, Sr, V, a rare-earth element, or a combination thereof, D is O, F, S, P, or a combination thereof, G is Al, Cr, Mn, Fe, Mg, La, Ce, Sr, V, or a combination thereof, and L¹ is Mn, Al, or a combination thereof.

For example, the positive electrode active material may be a high nickel-based positive electrode active material having a nickel amount of equal to or greater than about 80 mol %, equal to or greater than about 85 mol %, equal to or greater than about 90 mol %, equal to or greater than about 91 mol %, or equal to or greater than about 94 mol % and equal to or less than about 99 mol % relative to 100 mol % of metal devoid of lithium in the lithium transition metal composite oxide. The high nickel-based positive electrode active material may achieve high capacity and thus may be applied to a high-capacity and high-density (e.g., high energy density) rechargeable lithium battery.

### Negative Electrode 20

The negative electrode 20 for a rechargeable lithium battery may include a current collector COL2 and a negative electrode active material layer AML2 on the current collector COL2. The negative electrode active material layer AML2 may include a negative electrode active material and may further include a binder and/or a conductive material (e.g., an electrically conductive material).

For example, the negative electrode active material layer AML2 may include a negative electrode active material of about 90 wt % to about 99 wt %, a binder of about 0.5 wt % to about 5 wt %, and a conductive material (e.g., an electrically conductive material) of about 0 wt % to about 5 wt %.

The binder may serve to improve attachment of negative electrode active material particles to each other and also to improve attachment of the negative electrode active material to the current collector COL2. The binder may include a non-aqueous binder, an aqueous binder, a dry binder, or a combination thereof.

The non-aqueous binder may include polyvinyl chloride, carboxylated polyvinyl chloride, polyvinyl fluoride, ethylene propylene copolymer, polystyrene, polyurethane, polytetrafluoroethylene, polyvinylidene fluoride, polyethylene, polypropylene, polyamide imide, polyimide, or a combination thereof.

The aqueous binder may include styrene-butadiene rubber, (meth)acrylated styrene-butadiene rubber, (meth)acrylic rubber, butyl rubber, fluoro elastomer, polyethylene oxide, polyvinyl pyrrolidone, polyepichlorohydrin, polyphosphazene, poly(meth)acrylonitrile, ethylene propylene diene copolymer, polyvinyl pyridine, chlorosulfonated polyethylene, latex, polyester resin, (meth)acrylic resin, phenolic resin, epoxy resin, polyvinyl alcohol, or a combination thereof.

If an aqueous binder is used as the negative electrode binder, a cellulose-based compound capable of providing or increasing viscosity may further be included. The cellulose-based compound may include one or more selected from carboxymethyl cellulose, hydroxypropylmethyl cellulose, methyl cellulose, and alkali metal salts thereof. The alkali metal may include Na, K, and/or Li.

The dry binder may include a fibrillizable polymer material, for example, polytetrafluoroethylene, polyvinylidene fluoride, polyvinylidene fluoride-hexafluoropropylene copolymer, polyethylene oxide, or a combination thereof.

The conductive material may be used to provide an electrode with conductivity (e.g., electrical conductivity), and any suitable conductive material that does not cause a chemical change of a battery (e.g., does not cause an undesirable chemical change in the rechargeable lithium battery) may be used as the conductive material to constitute the battery. For example, the conductive material may include a carbon-based material such as natural graphite, artificial graphite, carbon black, acetylene black, Ketjenblack, carbon fiber, carbon nano-fiber, and/or carbon nano-tube; a metal powder and/or metal fiber including one or more of copper, nickel, aluminum, and/or silver; a conductive polymer (e.g., an electrically conductive polymer) such as a polyphenylene derivative; or a mixture thereof.

The current collector COL2 may include a copper foil, a nickel foil, a stainless-steel foil, a titanium foil, a nickel foam, a copper foam, a polymer substrate coated with a conductive metal (e.g., an electrically conductive metal), or a combination thereof.

### Negative Electrode Active Material

The negative electrode active material in the negative electrode active material layer AML2 may include a material that can reversibly intercalate and deintercalate lithium ions, lithium metal, a lithium metal alloy, a material that can dope and de-dope lithium, and/or transition metal oxide.

The material that can reversibly intercalate and deintercalate lithium ions may include a carbon-based negative electrode active material, for example, crystalline carbon, amorphous carbon, or a combination thereof. For example, the crystalline carbon may include graphite such as non-shaped, sheet-shaped, flake-shaped, sphere-shaped, and/or fiber-shaped natural or artificial graphite, and/or the amorphous carbon may include soft carbon, hard carbon, mesophase pitch carbon, and/or calcined coke.

The lithium metal alloy may include an alloy of lithium and metal that is selected from Na, K, Rb, Cs, Fr, Be, Mg, Ca, Sr, Si, Sb, Pb, In, Zn, Ba, Ra, Ge, Al, and Sn.

The material that can dope and de-dope lithium may include a Si-based negative electrode active material and/or a Sn-based negative electrode active material. The Si-based negative electrode active material may include silicon, silicon-carbon composite, SiOₓ (where 0<x<2), Si-Q alloy (where Q is alkali metal, alkaline earth metal, Group 13 element, Group 14 element (except for Si), Group 15 element, Group 16 element, transition metal, a rare-earth element, or a combination thereof), or a combination thereof. The Sn-based negative electrode active material may include Sn, SnO₂, a Sn-based alloy, or a combination thereof.

The silicon-carbon composite may be a composite of silicon and amorphous carbon. According to an embodiment, the silicon-carbon composite may have a structure in which the amorphous carbon is coated on a surface of the silicon particle. For example, the silicon-carbon composite may include a secondary particle (core) in which primary silicon particles are assembled (e.g., agglomerated), and an amorphous carbon coating layer (shell) on a surface of the secondary particle. The amorphous carbon may also be between the primary silicon particles, and for example, the primary silicon particles may be coated with the amorphous carbon. The secondary particles may be present dispersed in an amorphous carbon matrix.

The silicon-carbon composite may further include crystalline carbon. For example, the silicon-carbon composite may include a core including crystalline carbon and silicon particles and may also include an amorphous carbon coating layer on a surface of the core.

The Si-based negative electrode active material and/or the Sn-based negative electrode active material may be used in combination with a carbon-based negative electrode active material.

### Separator 30

Based on a type (or kind) of the rechargeable lithium battery, the separator 30 may be present between positive electrode 10 and the negative electrode 20. The separator 30 may include one or more selected from polyethylene, polypropylene, and polyvinylidene fluoride, and may have a multi-layered separator thereof such as a polyethylene/polypropylene bi-layered separator, a polyethylene/polypropylene/polyethylene tri-layered separator, and/or a polypropylene/polyethylene/polypropylene tri-layered separator.

The separator 30 may include a porous substrate and a coating layer on one or opposite surfaces of the porous substrate, which coating layer includes an organic material, an inorganic material, or a combination thereof.

The porous substrate may be a polymer layer including one selected from polyolefin such as polyethylene and polypropylene, polyester such as polyethylene terephthalate and polybutylene terephthalate, polyacetal, polyamide, polyimide, polycarbonate, polyetherketone, polyaryletherketone, polyetherimide, polyamideimide, polybenzimidazole, polyethersulfone, polyphenyleneoxide, cyclic olefin copolymer, polyphenylenesulphide, polyethylene naphthalate, glass fiber, Teflon, and polytetrafluoroethylene, or may be a copolymer or mixture including two or more of the materials mentioned above.

The organic material may include a polyvinylidenefluoride-based copolymer and/or a (meth)acrylic copolymer.

The inorganic material may include an inorganic particle selected from Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, Boehmite, or a combination thereof, but the present disclosure is not limited thereto.

The organic material and the inorganic material may be present mixed together in one coating layer or may be present as a stack of a coating layer including the organic material and a coating layer including an inorganic material.

### Electrolyte ELL

The electrolyte ELL for a rechargeable lithium battery may include a non-aqueous organic solvent and a lithium salt.

The non-aqueous organic solvent may serve as a medium that transmits ions that participate in an electrochemical reaction of a battery.

The non-aqueous organic solvent may include a carbonate-based solvent, an ester-based solvent, an ether-based solvent, a ketone-based solvent, an alcohol-based solvent, an aprotic solvent, or a combination thereof.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), and/or butylene carbonate (BC).

The ester-based solvent may include methyl acetate, ethyl acetate, n-propyl acetate, dimethyl acetate, methyl propionate, ethyl propionate, decanolide, mevalonolactone, valerolactone, and/or caprolactone.

The ether-based solvent may include dibutyl ether, tetraglyme, diglyme, dimethoxyethane, 2-methyltetrahydrofuran, 2.5-dimethyltetrahydrofuran, and/or tetrahydrofuran. The ketone-based solvent may include cyclohexanone. The aprotic solvent may include nitriles such as R—CN (where R is a hydrocarbon group having a C2 to C20 linear, branched, or cyclic structure and may include a double bond, an aromatic ring, and/or an ether group); amides such as dimethylformamide; dioxolanes such as 1,3-dioxolane and/or 1,4-dioxolane; and/or sulfolanes.

The non-aqueous organic solvent may be used alone or in a mixture of two or more substances.

In embodiments, if a carbonate-based solvent is used, a cyclic carbonate and a chain carbonate may be mixed together and used, and the cyclic carbonate and the chain carbonate may be mixed together in a volume ratio of about 1:1 to about 1:9.

The lithium salt may be a material that is dissolved in the non-aqueous organic solvent to serve as a supply source of lithium ions in a battery and plays a role in enabling a basic operation of a rechargeable lithium battery and in promoting the movement of lithium ions between positive and negative electrodes. The lithium salt may include, for example, at least one selected from LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li(FSO₂)₂N (lithium bis(fluorosulfonyl)imide, LiFSI), LiC₄F₉SO₃, LiN(CₓF₂ₓ₊₁SO₂)(C_{y}F_{2y+1}SO₂) (where x and y are integers between 1 to 20), lithium trifluoromethane sulfonate, lithium tetrafluoroethanesulfonate, lithium difluoro(oxalato)borate (LiDFOB), lithium difluorobis(oxalato)phosphate (LiDFBOP), and lithium bis(oxalato)borate (LiBOB).

### Rechargeable Lithium Battery

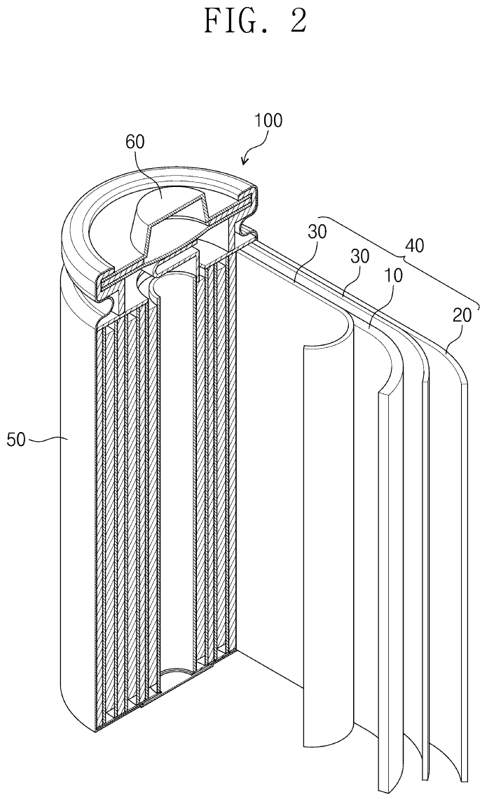

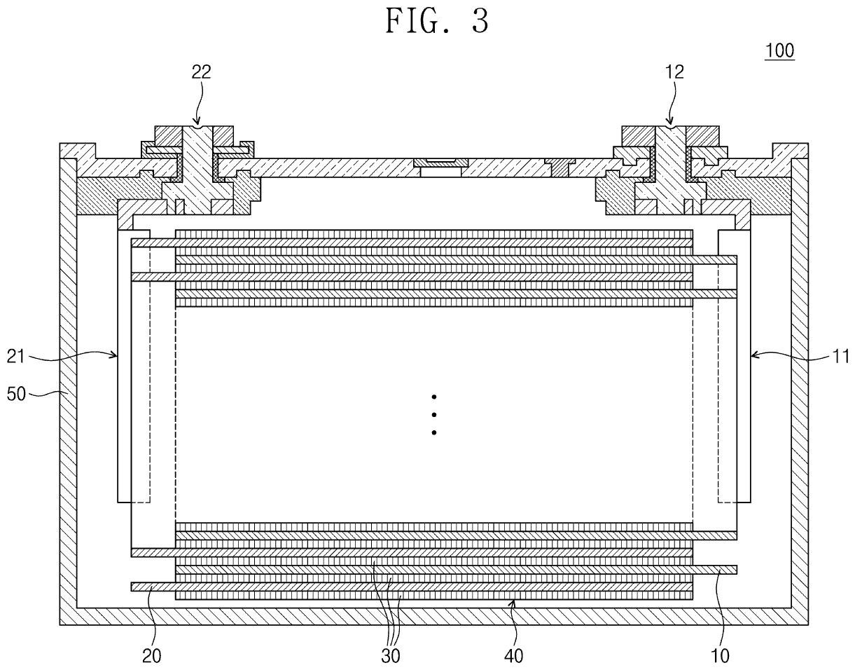

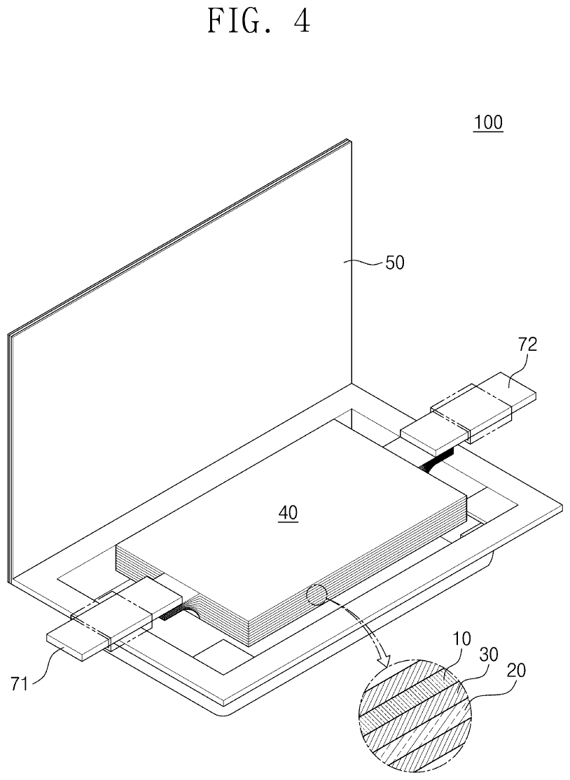

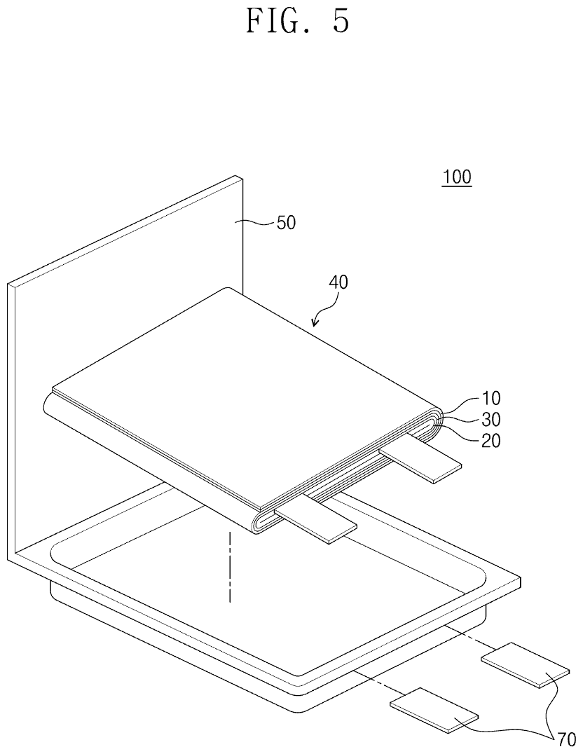

Based on a shape of a rechargeable lithium battery, the rechargeable lithium battery may be classified into cylindrical, prismatic, pouch, and/or coin types (or kinds). FIGS. 2-5 are simplified diagrams showing rechargeable lithium batteries according to embodiments, FIG. 2 shows a cylindrical battery, FIG. 3 shows a prismatic battery, and FIGS. 4-5 show pouch-type batteries. Referring to FIGS. 2-4, a rechargeable lithium battery 100 may include an electrode assembly 40 in which a separator 30 is between a positive electrode 10 and a negative electrode 20, and may also include a casing 50 in which the electrode assembly 40 is accommodated. The positive electrode 10, the negative electrode 20, and the separator 30 may be impregnated with an electrolyte. The rechargeable lithium battery 100 may include a sealing member 60 that seals the casing 50 as illustrated in FIG. 2. In embodiments, as illustrated in FIG. 3, the rechargeable lithium battery 100 may include a positive electrode lead tab 11, a positive electrode terminal 12, a negative electrode lead tab 21, and a negative electrode terminal 22. As shown in FIGS. 4-5, the rechargeable lithium battery 100 may include an electrode tab 70 (FIG. 5), or a positive electrode tab 71 and a negative electrode tab 72 (FIG. 4), which electrode tab 70 serves as an electrical path for externally inducing a current generated in the electrode assembly 40.

The following describes in more detail an electrolyte for a rechargeable lithium battery according to some embodiments of the present disclosure.

An electrolyte for a rechargeable lithium battery according to an embodiment may include a non-aqueous organic solvent, a lithium salt, and an additive.

The additive according to an embodiment of the present disclosure may be a material represented by Chemical Formula 1 below.

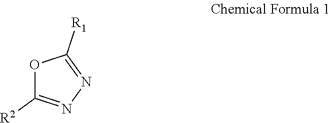

In Chemical Formula 1, R₁ may be a C1 to C10 alkyl group substituted with one or more fluorine atoms, and R₂ may be an aryl group.

The aryl group may be one selected from a phenyl group, a tolyl group, a xylenyl group, a naphthyl group, an anthracenyl group, a phenanthryl group, a naphthacenyl group, a pyrenyl group, a biphenylyl group, a terphenyl group, a chrycenyl group, a spirobifluorenyl group, a fluoranthenyl group, a fluorenyl group, a perylenyl group, an indenyl group, an azulenyl group, a heptalenyl group, a phenalenyl group, and a phenanthrenyl group.

For example, in Chemical Formula 1, R₂ may be a C6 to C15 aryl group, and R₂ may be one selected from a phenyl group, a tolyl group, a xylenyl group, and a naphthyl group.

Chemical Formula 1 may be represented by Chemical Formula 1-1 below.

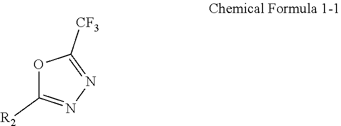

In Chemical Formula 1-1, R₂ may be an aryl group.

For example, Chemical Formula 1 may be represented by one selected from Chemical Formulae 1-2, 1-3, 1-4, and 1-5 below.

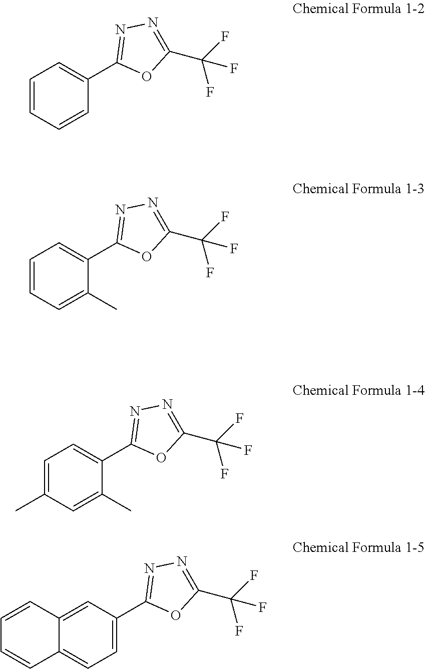 [SMILES: FC(F)(F)c1nnc(-c2ccccc2)o1 ; Cc1ccccc1-c1nnc(C(F)(F)F)o1 ; Cc1ccc(-c2nnc(C(F)(F)F)o2)c(C)c1 ; FC(F)(F)c1nnc(-c2ccc3ccccc3c2)o1]

The additive according to an embodiment of the present disclosure may be an oxadiazole-based compound including an aryl group and an alkyl group substituted with at least one fluorine atom.

As the additive has a halogen component such as a fluorine atom, LiF generated during charge and discharge may form a strong film on positive and negative electrodes. Thus, the additive may effectively contribute to lifetime characteristics of the lithium battery.

Because the additive contains an aryl group, the additive may be oxidized to form a film on a surface of the positive electrode through the generation of cathode-electrolyte interface (CEI), thereby inhibiting or reducing the decomposition of a positive electrode active material. Thus, it may be possible to suppress or reduce gas generation due to the decomposition of the positive electrode active material.

The additive may be included in an amount of about 0.01 wt % to about 10 wt % relative to the total amount of the electrolyte. For example, the additive may be included in an amount of about 0.5 wt % to about 3 wt % relative to the total weight of the electrolyte. If an amount of the additive is less than the range above, there may occur a problem where a film cannot be sufficiently formed on lithium-based positive and negative electrodes, and if an amount of the additive is greater than the range above, there may occur a problem where battery capacity and lifetime are reduced due to an increase in resistance (e.g., electrical resistance) of the positive and negative electrodes.

The electrolyte may be manufactured by a mixing process in which the lithium salt is dissolved in the non-aqueous organic solvent and the additive is added to the resultant mixture. The electrolyte mixing process may be any suitable one generally used in the electrolyte fabrication field, and a person having ordinary skill in the art should be able to suitably or appropriately select and use the electrolyte mixing process upon reviewing this disclosure.

The non-aqueous organic solvent may include at least one selected from ethylmethyl carbonate (EMC), ethylene carbonate (EC), dimethyl carbonate (DMC), propylene carbonate (PC), propyl propionate (PP), diethyl carbonate (DEC), dipropyl carbonate (DPC), methylpropyl carbonate (MPC), ethylpropyl carbonate (EPC), methylethyl carbonate (MEC), and butylene carbonate (BC).

In an embodiment, the non-aqueous organic solvent may be a mixed solvent of ethylmethyl carbonate (EMC), ethylene carbonate (EC), and dimethyl carbonate (DMC).

The ethylmethyl carbonate (EMC), the ethylene carbonate (EC), and the dimethyl carbonate (DMC) may have a volume ratio of 1:a:b, The a may be about 1 to about 3, and the b may be about 5 to about 8.

For example, the ethylmethyl carbonate (EMC) may be included in an amount of about 5 vol % to about 20 vol % relative to the total volume of the non-aqueous organic solvent. The ethylene carbonate (EC) may be included in an amount of about 10 vol % to about 30 vol % relative to the total volume of the non-aqueous organic solvent. The dimethyl carbonate (DMC) may be included in an amount of about 60 vol % to about 80 vol % relative to the total volume of the non-aqueous organic solvent.

The lithium salt may include at least one selected from LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, Li(FSO₂)₂N, lithium bis(fluorosulfonyl)imide (LiFSI), and LiC₄F₉SO₃. According to an embodiment, the lithium salt may include LiPF₆.

The lithium salt may have a concentration of about 0.1 M to about 2.0 M. For example, the lithium salt may have a concentration of equal to or greater than about 0.5 M or about 1.0 M. The lithium salt may have a concentration of equal to or less than about 2.0 M, equal to or less than about 1.7 M, or equal to or less than about 1.5 M. In the present disclosure, if the lithium salt has a concentration of about 0.1 M to about 2.0 M, the electrolyte may suitably or appropriately maintain its conductivity (e.g., electrical conductivity) and viscosity.

In an embodiment of the present disclosure, a rechargeable lithium battery may include a positive electrode that includes a positive electrode active material, a negative electrode that includes a negative electrode active material, and an electrolyte, and the electrolyte may include a non-aqueous organic solvent, a lithium salt, an additive represented by Chemical Formula 1 above.

The positive electrode active material of the rechargeable lithium battery may include one or more of cobalt-free nickel-manganese-based oxide, lithium-nickel-based oxide, lithium-cobalt-based oxide, lithium-manganese-based oxide, lithium-iron-phosphate-based compounds, and/or any combination thereof. For example, the positive electrode active material may include cobalt-free nickel-manganese-based oxide.

In a rechargeable lithium battery using an electrolyte according to the present disclosure, a negative electrode active material may include a carbon-based negative electrode active material, a silicon-based negative electrode active material, or any combination thereof.

The silicon-based negative electrode active material may include a core including silicon-based particles and a coating layer including amorphous carbon. The silicon-based particles may include one or more selected from a silicon particle, a silicon-carbon composite, SiOₓ (where 0<x≤2), and a silicon alloy.

The rechargeable lithium battery may operate even at high voltages of equal to or greater than about 4.5 V.

The rechargeable lithium battery may be applied to automotive vehicles, mobile phones, and/or any other suitable electrical devices, but the present disclosure is not limited thereto.

The following will describe Embodiments and Comparative Example 1 of the present disclosure. The following Embodiments, however, are merely examples, and the present disclosure is not limited to Embodiments discussed below.

### Embodiments and Comparative Example 1

### Embodiment 1

### (1) Preparation of Electrolyte

1.5 M LiPF₆ was dissolved in a non-aqueous organic solvent including ethylene carbonate (EC), ethylmethyl carbonate (EMC), and dimethyl carbonate (DMC) mixed together in a volume ratio of about 20:10:70, and an additive of 0.5 wt % was added to prepare an electrolyte.

A material represented by Chemical Formula 1-2 below was used as the additive.

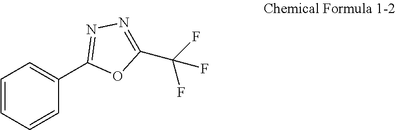 [SMILES: FC(F)(F)c1nnc(-c2ccccc2)o1]

### (2) Fabrication of Rechargeable Lithium Battery

97 wt % of LiNiMnO (NMX) as a positive electrode active material, 0.5 wt % of artificial graphite powder as a conductive material, 0.8 wt % of carbon black (Ketjenblack), 0.2 wt % of acrylonitrile rubber, 1.5 wt % of polyvinylidene fluoride (PVdF) were mixed together and added to N-methyl-2-pyrrolidone, and then the resultant mixture was stirred for 30 minutes by using a mechanical agitator to prepare a positive electrode active material slurry. A doctor blade was used to coat the slurry to a thickness of about 60 μm on an aluminum current collector of about 20 μm, dried in a hot-air drier for 0.5 hours at 100° C., dried again for 4 hours at 120° C. under a vacuum condition, and then roll-pressed to manufacture a positive electrode.

98 wt % of a negative electrode active material containing artificial graphite and silicon composite mixed together in a volume ratio of 93:7, 1 wt % of styrene-butadiene rubber (SBR), and 1 wt % of carboxymethyl cellulose (CMC) were mixed together and added to distilled water, and then stirred for 60 minutes by using a mechanical agitator to prepare a negative electrode active material slurry. A doctor blade was used to coat the slurry to a thickness of about 60 μm on a copper current collector of about 10 μm, dried in a hot-air drier for 0.5 hours at 100° C., dried again for 4 hours 120° C. under a vacuum condition, and then roll-pressed to manufacture a negative electrode.

The positive electrode, the negative electrode, and a polyethylene separator of 10 μm in thickness were assembled to manufacture an electrode assembly, and the electrolyte was introduced to fabricate a rechargeable lithium battery.

### Embodiment 2

An electrolyte and a rechargeable lithium battery were fabricated according to substantially the same method as in Embodiment 1, except that 1.0 wt % of additive was applied.

### Embodiment 3

An electrolyte and a rechargeable lithium battery were fabricated according to substantially the same method as in Embodiment 1, except that 3.0 wt % of an additive was applied.

### Embodiment 4

An electrolyte and a rechargeable lithium battery were fabricated according to substantially the same method as in Embodiment 1, except the use of an additive represented by Formula 1-3 below.

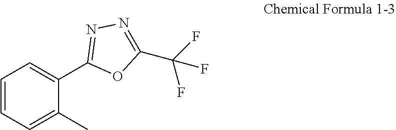 [SMILES: Cc1ccccc1-c1nnc(C(F)(F)F)o1]

### Embodiment 5

An electrolyte and a rechargeable lithium battery were fabricated according to substantially the same method as in Embodiment 1, except the use of an additive represented by Formula 1-4 below.

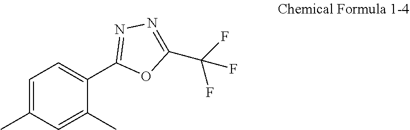 [SMILES: Cc1ccc(-c2nnc(C(F)(F)F)o2)c(C)c1]

### Embodiment 6

An electrolyte and a rechargeable lithium battery were fabricated according to substantially the same method as in Embodiment 1, except the use of an additive represented by Formula 1-5 below.

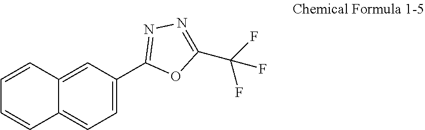 [SMILES: FC(F)(F)c1nnc(-c2ccc3ccccc3c2)o1]

### Comparative Example 1

An electrolyte and a rechargeable lithium battery were fabricated according to substantially the same method as in Embodiment 1, except the use of an additive represented by Chemical Formula 1-2 above if the electrolyte was prepared.

### Evaluation Example

A rechargeable lithium battery was evaluated by the following method.

### Evaluation 1: CV Characteristics

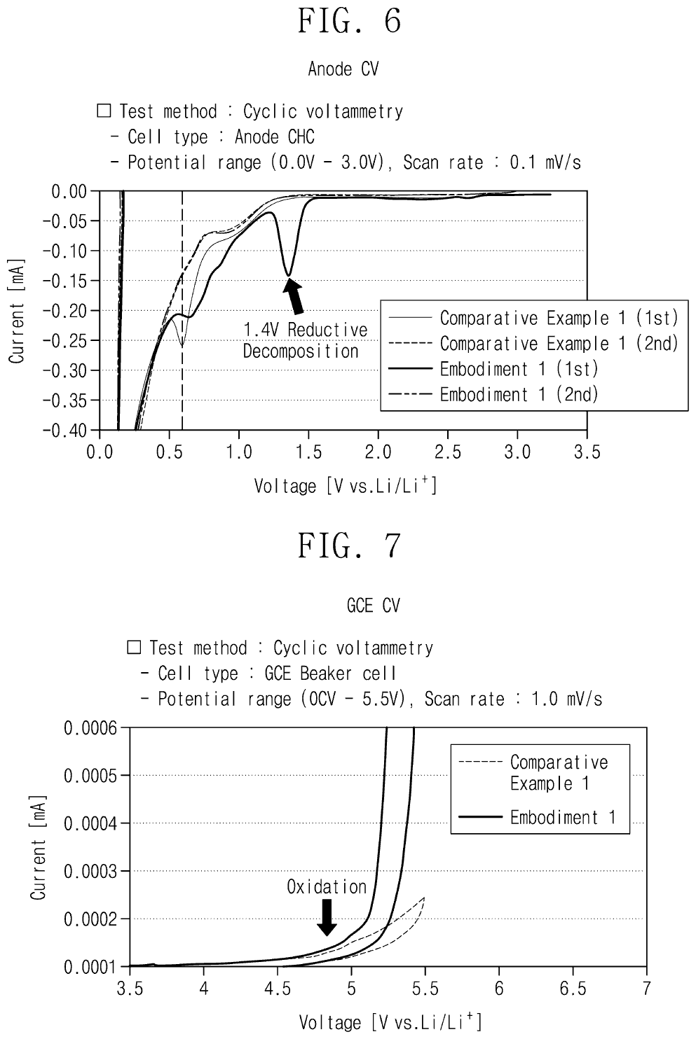

A cyclic voltammetry (CV) was measured at room temperature (25° C.) to evaluate electrochemical stability of the electrolytes used in Embodiment 1 and Comparative Example 1, and the measured results are depicted in FIGS. 6-7. FIG. 6 is a graph showing a result of negative electrode cyclic voltammetry (CV) according to Embodiment 1 and Comparative Example 1. FIG. 7 is a graph showing a result of cyclic voltammetry (CV) of glassy carbon electrodes (GCE) according to Embodiment 1 and Comparative Example 1.

A negative electrode cyclic voltammetry (CV) was measured by using a coin half-cell in which a graphite negative electrode was used as a working electrode and Li metal is used as a counter electrode. In this measurement, a scan was performed at a rate of 0.1 mV/see in a range from 3.0 V to 0 V.

A cyclic voltammetry (CV) of glassy carbon electrodes (GCE) was measured by using a coin half-cell in which a glassy carbon electrode (GCE) is used as a working electrode, a platinum (Pt) wire was used as a counter electrode, and a saturated calomel electrode was used as a reference electrode.

In this measurement, a scan was performed at a rate of 0.1 mV/see in a range from 5.0 V to an open circuit voltage (OCV).

### Evaluation 2: High-Temperature Storage Characteristics (DC-IR Change Rate)

The rechargeable lithium batteries fabricated according to the Embodiments and Comparative Example 1 were measured with respect to ΔV/ΔI (voltage change/current change) as initial direct current internal resistance (DC-IR), direct current internal resistances (DC-IR) were measured by allowing the batteries to change their internal maximum energy states into full-charge states (SOC 100%), and in this stage, the batteries were stored at a high temperature (60° C.) for 30 days to calculate DC-IR increase rates (%) according to Equation 1 below and to list the calculated results in Table 1 below.

$$DC ‐ IR increase rate = ( DC ‐ IR after 30 days / initial DC ‐ IR ) \times 100 \qquad Equation 1$$

TABLE 1

| | Initial | DC-IR after storage for 30 | DC-IR increase |
|---|---|---|---|
| | DC-IR | days at 60° C. | rate |
| Category | mΩ | mΩ | % |
| Comparative | 42.28 | 52.00 | 123 |
| Example 1 | | | |
| Embodiment 1 | 42.30 | 50.04 | 118 |
| Embodiment 2 | 42.32 | 49.74 | 117 |
| Embodiment 3 | 42.55 | 52.11 | 122 |
| Embodiment 4 | 42.42 | 52.09 | 123 |
| Embodiment 5 | 42.28 | 52.12 | 123 |
| Embodiment 6 | 42.78 | 52.45 | 123 |

### Evaluation 3: Gas Generation at High Temperature

The rechargeable lithium batteries according to the Embodiments and Comparative Example 1 underwent evaluation of gas generation characteristics at high temperatures. The rechargeable lithium batteries according to the Embodiments and Comparative Example 1 were charged to 4.53 V at 45° C. and then left for 30 days at 60° C.

To ascertain a gas reduction effect, an initial thickness of the cell battery and a thickness of the cell battery after 7 days were each measured, and results are listed in Table 2 below.

TABLE 2

Gas generation amount at high temperature
(60° C.) (mL)

| Category | 1^{st} day | 7^{th} day |
|---|---|---|
| Comparative | 0.033 | 0.080 |
| Example 1 | | |
| Embodiment 1 | 0.033 | 0.069 |
| Embodiment 2 | 0.033 | 0.045 |
| Embodiment 3 | 0.033 | 0.041 |
| Embodiment 4 | 0.031 | 0.054 |
| Embodiment 5 | 0.031 | 0.052 |
| Embodiment 6 | 0.029 | 0.047 |

### Evaluation 4: High-Temperature Charge/Discharge Cycle Characteristics

The rechargeable lithium batteries fabricated in the Embodiments and Comparative Example 1 were charged and discharged at 60° C. for 200 cycles under the conditions of 2.0 C charge (CC/CV, 4.53 V Cut-off) and 1.0 C discharge (CC, 3.0 V Cut-off), and high-temperature capacity retention rates were calculated and listed in Table 3 below. The high-temperature capacity retention rate was calculated according to Equation 2 below.

$$High ‐ temperature capacity retention rate ( \% ) = ( discharge capacity after 200 cycles / initial discharge capacity ) \times 100 \qquad Equation 2$$

TABLE 3

| | High-temperature capacity retention |
|---|---|
| Category | rate (%) |
| Comparative | 89.1 |
| Example 1 | |
| Embodiment 1 | 93.7 |
| Embodiment 2 | 94.5 |
| Embodiment 3 | 94.1 |
| Embodiment 4 | 95.1 |
| Embodiment 5 | 94.8 |
| Embodiment 6 | 93.7 |

### Comprehensive Evaluation

Referring to FIG. 6, it may be ascertained that, compared to Comparative Example 1, the electrolyte according to Embodiment 1 exhibits a reductive decomposition peak at a higher voltage. Thus, it may be expected that the electrolyte according to Embodiment 1 causes the formation of an initial solid electrolyte interface (SEI) film on the negative electrode before the occurrence of solvent decomposition during a charge procedure in which lithium ions are intercalated into the negative electrode.

Referring to FIG. 7, it may be ascertained that oxidation occurs at a lower voltage in the electrolyte according to Embodiment 1 compared to Comparative Example 1. Thus, the aryl group of the additive in the electrolyte according to Embodiment 1 may be oxidized to generate a cathode-electrolyte interface (CEI) to form a film on the positive electrode, and accordingly, the electrolyte according to Embodiment 1 may improve battery performance and lifetime.

Referring to Table 1, it may be observed that there is an improvement in high-temperature storage (60° C.) in cases (Embodiments 1 to 6) each using an electrolyte with the additive according to the present disclosure, compared to a case (Comparative Example 1) using an electrolyte without any additive.

Referring to Table 2, it may be observed that the rechargeable lithium battery fabricated according to Comparative Example 1 has a large amount of gas generation at high-temperature storage (60° C.) compared to the rechargeable lithium batteries fabricated according to the Embodiments. Therefore, the gas generation at a high temperature (60° C.) may be effectively suppressed or reduced in the rechargeable lithium battery that uses the additive represented by Chemical Formula 1 according to embodiments of the present disclosure.

Referring to Table 3, it may be observed that battery cycle characteristics and lifetime efficiency under the environment of high-temperature storage (60° C.) are improved in a case using an electrolyte with the additive according to embodiments of the present disclosure than in a case using an electrolyte without any additive.

An electrolyte according to an embodiment may include an additive containing oxadiazole with which a fluoroalkyl group and an aryl group are combined, and thus lifetime characteristics and stability under the condition of high temperature may be improved if rechargeable batteries including the electrolyte are activated.

While the subject matter of this disclosure has been described in connection with what is presently considered to be example embodiments, it is to be understood that the present disclosure is not limited to the disclosed embodiments and is intended to cover various modifications and equivalent arrangements included within the spirit and scope of the appended claims and equivalents thereof, and therefore the aforementioned embodiments should be understood to be examples but not limiting this disclosure in any way.

What is claimed is:

1. An electrolyte for a rechargeable lithium battery, the electrolyte comprising:
a non-aqueous organic solvent;
a lithium salt; and
an additive represented by Chemical Formula 1, 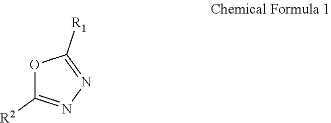
wherein, in Chemical Formula 1,
R₁ is a C1 to C10 alkyl group substituted with one or more fluorine atoms, and
R₂ is a C6 to C15 aryl group.

2. The electrolyte as claimed in claim 1, wherein R₂ is one selected from a phenyl group, a tolyl group, a xylenyl group, and a naphthyl group.

3. The electrolyte as claimed in claim 1, wherein the additive is represented by Chemical Formula 1-1, 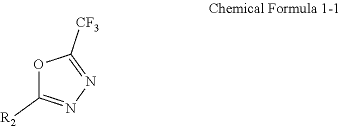
wherein, in Chemical Formula 1-1, R₂ is an aryl group.

4. The electrolyte as claimed in claim 1, wherein the additive is represented by one selected from Chemical Formulae 1-2, 1-3, 1-4, and 1-5,

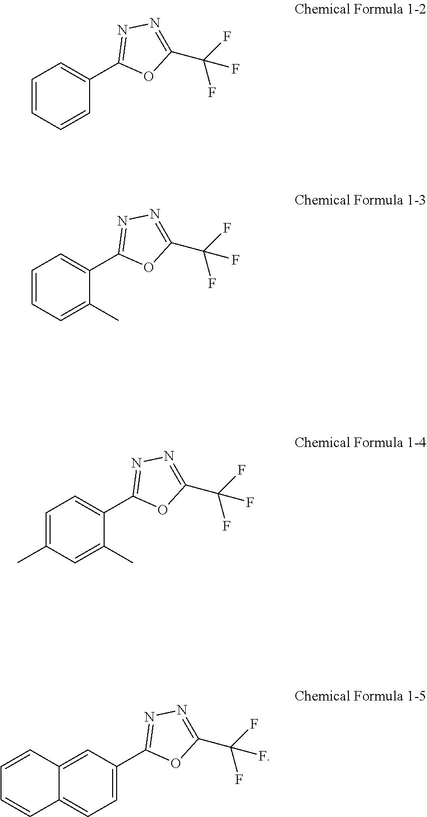 [SMILES: FC(F)(F)c1nnc(-c2ccccc2)o1 ; Cc1ccccc1-c1nnc(C(F)(F)F)o1 ; Cc1ccc(-c2nnc(C(F)(F)F)o2)c(C)c1 ; FC(F)(F)c1nnc(-c2ccc3ccccc3c2)o1]

5. The electrolyte as claimed in claim 1, wherein the additive is present in an amount of about 0.01 wt % to about 10 wt % relative to a total weight of the electrolyte.

6. The electrolyte as claimed in claim 1, wherein the non-aqueous organic solvent comprises at least one selected from ethylmethyl carbonate (EMC), ethylene carbonate (EC), dimethyl carbonate (DMC), propylene carbonate (PC), propyl propionate (PP), diethyl carbonate (DEC), dipropyl carbonate (DPC), methylpropyl carbonate (MPC), ethylpropyl carbonate (EPC), methylethyl carbonate (MEC), and butylene carbonate (BC).

7. The electrolyte as claimed in claim 1, wherein the non-aqueous organic solvent comprises ethylmethyl carbonate (EMC), ethylene carbonate (EC), and dimethyl carbonate (DMC),
wherein the ethylmethyl carbonate (EMC), the ethylene carbonate (EC), and the dimethyl carbonate (DMC) have a volume ratio of 1:a:b,
wherein a is about 1 to about 3, and
wherein b is about 5 to about 8.

8. The electrolyte as claimed in claim 1, wherein the lithium salt comprises at least one selected from LiPF₆, LiBF₄, LiSbF₆, LiAsF₆, LiClO₄, LiAlO₂, LiAlC₄, LiPO₂F₂, LiCl, LiI, LiN(SO₃C₂F₅)₂, Li(FSO₂)₂N, lithium bis(fluorosulfonyl)imide (LiFSI), and LiC₄F₉SO₃.

9. The electrolyte as claimed in claim 1, wherein a concentration of the lithium salt is in a range of about 0.1 M to about 2.0 M.

10. A rechargeable lithium battery, comprising:
a positive electrode comprising a positive electrode active material;
a negative electrode comprising a negative electrode active material; and
the electrolyte for the rechargeable lithium battery as claimed in claim 1.

11. The rechargeable lithium battery as claimed in claim 10, wherein the positive electrode active material comprises cobalt-free nickel-manganese-based oxide, lithium-nickel-based oxide, lithium-cobalt-based oxide, lithium-manganese-based oxide, lithium-iron-phosphate-based compounds, or any combination thereof.

12. The rechargeable lithium battery as claimed in claim 10, wherein the negative electrode active material comprises a carbon-based negative electrode active material, a silicon-based negative electrode active material, or a combination thereof.

13. The rechargeable lithium battery as claimed in claim 12, wherein the silicon-based negative electrode active material comprises a silicon-carbon composite.

14. The rechargeable lithium battery as claimed in claim 10, wherein the rechargeable lithium battery operates even at a high voltage of equal to or greater than about 4.5 V.
